(19) United States
(12) Patent Application Publication
(10) Pub. No.: US 2011/0311622 A1
(43) Pub. Date: Dec. 22, 2011

(54) METHOD FOR ADDING SENSORY CONDITIONING CUES IN A PHARMACOTHERAPEUTIC REGIMEN

(76) Inventors: Joshua D. Levine, Chapel Hill, NC (US); Robert A. Levine, Guilford, CT (US)

(21) Appl. No.: 13/141,594

(22) PCT Filed: Dec. 22, 2009
(86) PCT No.: PCT/US09/69245

§ 371 Date: Sep. 8, 2011

Related U.S. Application Data
(60) Provisional application No. 61/140,447, filed on Dec. 23, 2008.

(51) Int. Cl. A61K 9/48; A61K 9/20; A61K 36/00; A61K 9/28

(52) U.S. Cl. 424/463; 424/474; 424/451; 424/464; 424/725

(57) ABSTRACT

A method for administering pharmaceutical agents to a subject is provided. The method includes the steps of: a) providing a course of treatment for a subject, which course includes periodically administering an amount of medicine to the subject, which amount of medicine includes a dose of at least one active pharmacological agent (APA) and an amount of at least one non-active pharmacological agent (NPA), which NPA provides at least one non-visual sensory cue; and b) varying the dosage amount of the APA within the periodically administered amount of medicine, while the amount of the NPA contained within each periodically administered amount of medicine is provided at a level that maintains the sensory cue at a substantially constant level.

This patent application claims priority from U.S. Provisional Application No. 61/140,447 filed Dec. 23, 2008, which is hereby incorporated by reference in its entirety.

### BACKGROUND OF THE INVENTION

1. Technical Field

The present invention describes a method for administering active pharmaceuticals (chemically synthesized, bioengineered, naturally occurring, or botanically derived) to a patient during a course of treatment. In particular, the present method makes use of the ‘placebo response’, and enhances it through the conditioning achieved by combining initial therapeutic pharmacological levels of the active pharmaceutical agent with “inactive” conditioning agents that contain a sensory cue. The term “inactive” means that the agent is not known to have significant pharmacological effects. The term “sensory conditioning cue” refers to both conscious conditioning and/or subconscious conditioning. Subconscious conditioning may occur when a subject encounters a stimulus, such as an ingested substance with a smell or taste, that the subject does not consciously perceive.

2. Background Information

It has been shown in numerous studies that when patients believe they are receiving a potent medication (even though the “potent” medication contained no active pharmacological ingredients) they will heal more rapidly and/or feel better more quickly and/or more fully than patients who are not given such treatment. The administration of a “placebo” (i.e., a non-active substitute for an active pharmacological agent) can activate physiological responses similar to those experienced by a patient receiving an active pharmacological agent. While the “placebo response” may not be entirely understood by modern science, it is well documented that rather than simply convincing patients into believing that they are getting better, improving, or healed, the so-called “placebo” can actually effect a scientifically documentable improvement or cure. In many studies of pharmacological efficacy the “active drug” performs only marginally better or moderately better, and in some cases no better, or worse than the placebo. Physicians must often weigh whether this small additional level of improvement is worth the concurrent risk of side effects caused by active pharmaceuticals. Placebos, by definition have little or no side effects since they are composed of substances generally recognized as safe and inactive. The use of placebos is widespread in both traditional and modem medicine and arguably forms the foundation of some forms of medical therapy.

The use of non-pharmacologically active (NPA) substances to effect a cure is highly desirable for a variety of reasons, including the fact that NPAs typically: 1) have less or no side effects; 2) cost less; 3) have less or no interaction with other medications; 4) have a decreased chance of addiction; and 5) have less or no side effects and/or complications associated with usage over an indefinite time period.

There are, however, a number of difficulties presented to a doctor wishing to treat a patient with a placebo. For example, a patient who enrolls in a double-blind study will be informed that they may be given a placebo treatment as part of their participation in the study. Absent this informed consent of the possibility of taking placebos, there are potentially serious ethical dilemmas associated with a doctor telling a patient he is receiving an active pharmacological agent (APA) when he is in fact being given a placebo.

### SUMMARY OF THE INVENTION

The current invention provides a method for administering an amount of medicine containing an active pharmaceutical agent and a placebo combined in a unitary dose form. The efficacy of the placebo is augmented by coupling it with the active pharmacologic agent, and by administering in a form that has a detectable sensory effects (e.g., taste, smell, sound, oropharyngeal somatosensory sensation, etc.). The somatic senses include the sensations of touch, pressure, temperature, nociception (pain) and proprioception. Oropharyngeal somatosensory sensations are somatic sensations perceived in the oropharynx, the region extending from the uvula to the hyoid bone.

According to an aspect of the present invention, a method for administering pharmaceutical agents to a subject is provided. The method includes the steps of: a) providing a course of treatment for a subject, which course includes periodically administering an amount of medicine to the subject, which amount of medicine includes a dose of at least one active pharmacological agent (APA) and an amount of at least one non-active pharmacological agent (NPA), which NPA provides at least one non-visual sensory cue; and b) varying the dosage amount of the APA within the periodically administered amount of medicine (e.g., below the generally prescribed dosage of the APA), while the amount of the NPA contained within each periodically administered amount of medicine is provided at a level that maintains the sensory cue at a substantially constant level.

In some embodiments, the physician may continue to administer the combination of the NPA and APA, with the APA at the lowest effective level for the patient and the NPA at a constant original level, thereby minimizing side effects of the APA that may be dose dependent. The APA dose level may be at any level within the generally recommended dose levels to levels that are thought to be pharmacologically inactive; the APA may be administered at a dosage that is the lowest dosage that will be effective for the patient taking the combined APA and NPA of the present invention.

One of the advantages provided by the present method is that the sensory cue(s), of perceived taste (such as that of cinnamon or chocolate), tingling (such as that induced by echinacea), sense of heat/minor burning sensation (such as that induced by capsaicin or ginger), sense of cooling (such as that induced by menthol) associated with the APA is stronger and longer lasting and much more certain to be perceived than a visual cue. It is known in the prior art to use a placebo dose of medicine within a prescribed course of treatment. For example, U.S. Pat. No. 6,855,324 “Therapeutic Placebo Enhancement of Commonly-Used Medications” describes a method for reducing the normal dosage of a pharmaceutical that involves the use of visual indicia associated the use of a full dosage and with a placebo. At a certain point in the treatment, a reduced dosage of the pharmaceutical is used with the placebo. Problems with using a visual indicia include the fact that they are only discernible prior to being ingested, they require the subject to be able to see them, and once ingested they are gone—nothing remains to reinforce the association between the visual cue and the medicine with which it is associated. Equally problematic is the fact that many medications are taken together and or administered to the patient by others and visual cues are therefore non-effective. The present invention, in contrast, uses a sensory cue that can remain with the subject for a period of time, does not rely on the subject's ability to see, and will be substantially more noticeable that a visual cue; e.g., a bitter flavor or tingling sensation can stay in the subject's mouth for a brief period of time to reinforce the desired association with the APA.

Another advantage of the present invention is that the present method for administering an amount of medicine capitalizes on the reassurance provided to a patient by his or her physician. The present coupling of a placebo with an active pharmacologic agent, which coupling generally requires a prescription or contact with his or her healthcare provider and therefore requires enhanced contact between the physician and patient, further augments the placebo response experienced by the patient. Coupling the placebo and the APA also avoids disclosure difficulties associated with a doctor wishing to treat a patient with a placebo. The co-administration of a placebo together with an APA resolves the difficulties since the patient is in fact receiving an APA. The combination described in the present invention will allow the physician to use the lowest possible dosage of APA thereby minimizing the dose related side effects of the APA.

Subjects most likely to benefit by the present method include those that are taking an APA for pain, mood disorders, anxiety, high blood pressure, depression, asthma, allergies, as well as symptoms triggered by chemical sensitivities. It will also benefit many other conditions including those with a possible psychosomatic trigger such as irritable bowel syndrome, sexual dysfunction, eating disorders including obesity, back pain, and various phobias.

The present apparatus and advantages associated therewith will become more readily apparent in view of the detailed description provided below.

### DETAILED DESCRIPTION OF THE INVENTION

According to the present invention, a method for periodically administering a medicine containing one or more active pharmaceutical agents (chemically or biologically synthesized, or botanically derived) to a patient within a prescribed course of treatment is provided. The periodically administered medicine may assume a variety of different forms (e.g., pill, capsule, tablet, powder, cream, patches, liquid, gas, etc), depending upon the application at hand. Hereinafter, the form of the medicine will be referred to hereinafter as a “pill” for ease of description. The periodically administered medicine is not, however, limited to only a solid “pill” form.

The one or more active pharmacological agents (APAs) within the pill are specifically chosen to create a desired effect on the subject taking the medicine. In addition to the APAs, each pill also includes an amount of one or more non-active pharmacological agents (NPAs). The NPAs include a composition that is, individually or collectively, capable of giving the patient a conscious (perceptible) and/or unconscious (imperceptible) cue (referred to hereinafter as a “sensory cue”) to its presence. A sensory cue may be perceptible prior to or at the time of ingestion or application; e.g., cues such as smell, taste, tactile texture, mouth sensation, and involuntary response (e.g., salivation). Sensory cues induced by stimulating the somatosensory system of the tongue and mouth include sensations such as burning, tingling, hotness, coolness, astringency, carbonation, mouthfulness (heartiness/kokumi), numbness, etc., can be particularly effective because of the response they create; i.e., one that can be stronger, longer lasting, and distinctive relative to sensory cues that a patient is likely to normally encounter. A patient having a dull sense of taste would likely readily recognize a tingling sensation. A sensory cue caused by an NPA may also be perceptible after ingestion; cues such as aftertaste, sweating, bodily waste coloration or odor, etc.

The NPAs that create a sensory cue also create and/or reinforce (by conscious and/or subconscious mechanisms) an association between the cue and the effect associated with the APA. In some embodiments, the association between the cue and the APA effect is created when the subject is treated with the medicine containing the APA over a period of time, and the sensory cue accompanying the APA becomes associated with the relief (i.e., effect) provided by the APA over the period of time. For example, under the present invention a pill containing a prescribed dosage of an anti-inflammatory agent could contain a specific amount of an NPA that imparts an arbitrarily chosen flavor, mouth sensation or has a distinct smell. After a period of time, that flavor, mouth sensation or smell will become associated with the anti-inflammatory relief provided by the APA. As a result, the subject will expect the anti-inflammatory relief, or be conditioned to experience the relief, when a pill is taken having the particular flavor, sensation or smell.

In other embodiments, the association between the sensory cue and the APA effect is a naturally occurring one (i.e., one that is independent of the medicine) that is reinforced when the subject is treated with medicine containing the APA and a NPA that imparts a particular sensory cue. For example, a pill containing a prescribed dosage of a strong anti-inflammatory agent may contain a specific amount of an NPA that contains or mimics the flavor and mouth sensation of capsaicin (chili pepper), which flavor is associated with anti-inflammatory relief. If the NPA actually contains capsaicin, the amount of the capsaicin is less than an amount that would provide anti-inflammatory relief to the subject by itself, but is an amount that is sufficient to provide the flavor and sensation associated with capsaicin. Hence, the anti-inflammatory relief provided by the medicine is naturally associated with the taste and sensation of the capsaicin (chili pepper), but it is the APA that provides the anti-inflammatory relief—not the capsaicin sensory cue associated with the aforesaid relief. Other examples of combinations of a sensory cue associated with particular relief and an APA that provides such relief include, but are not limited to: a) licorice or peppermint associated with an APA that restores calm breathing; b) chamomile flavor associated with an APA that helps induce sleep or calms anxiety; c) ginger, peppermint, or fennel associated with an APA that helps dissipate or prevent motion sickness; d) peppermint or chamomile associated with an APA that provides relief for irritated bowel syndrome; e) opiate alkaloids or opium-less and alkaloid-free seed poppy cultivar associated with an APA that provides pain relief; f) Echinacea alkymides and garlic associated with an APA that is an antiviral; g) licorice or coffee associated with an APA that provides asthma relief; h) lemon balm, chocolate, or bitters associated with an APA that provides depression relief; i) bergamot associated with an APA that provides relief for obsessive compulsive disorder; and j) chamomile or licorice associated with an APA used to treat ulcers.

For medicines that are, or were originally, directly or indirectly botanically derived, the NPA may contain non-active constituents of the plant from which it was derived. These non-active constituents, with nearly identical chemical compositions, may trigger unconscious perceptions/cues and enhance the effect of the NPA treatment regimen. For example, in certain applications an APA such as caffeine may be administered as treatment of various disorders and conditions (i.e., migraines, fatigue). In such applications, decaffeinated coffee (i.e., a non-active constituent derived from a botanical source) can provide both conscious and unconscious sensory cues that can enhance the effect of the NPA treatment regimen.

In some embodiments, a synthetically derived APA may be combined with one or more sensory cues of analogous plant medicines that produce the same pharmacological actions. For example, a synthetic opioid may taste strongly bitter and may numb the mouth like the Papaver somniferum alkaloid from which it was originally derived. A medication for flatulence or colic may have a fennel (Foeniculum vulgare) smell. A medication for ulcerative conditions of the bowels may have a peppermint smell which is known to have relaxing, anti-inflammatory effects on the muscles of the digestive system. A medicine to treat microbial infections may include the alkylamides of Echinacea spp. that produce a strong tingling sensation on the tongue. The sensory cues (both conscious and unconscious) assist the body in “understanding” and reacting to the medicine. They enhance the effect of the non-active pharmacological agents by providing memorable associative cues with the medication containing the active pharmacological agents.

In some embodiments, the NPA that creates or causes the sensory cue is admixed with the APA within a particular form; e.g., a pill, liquid, gel, etc.

In other embodiments, the NPA that creates or causes the sensory cue is provided in a form where it is not admixed with the APA. For example, the NPA could be provided as a coating on materials that include the APA; e.g., a coating on a pill, etc. Alternatively, the NPA could be incorporated into, or coated onto a capsule that is used to contain materials including the APA. For example, it is known to use gelatin capsules to “package” medicines, which “package” can be ingested. In many instances, but not all, the gelatin capsules are purposefully non-flavored. In other instances, however, the capsules are purposely given a flavor designed to increase the palatability of the capsule. U.S. Pat. No. 6,346,231 “Flavored Gelatin Capsule and Method of Manufacture” discloses such a gelatin based capsule, and is incorporated by reference herein in its entirety. The flavor is chosen for solely for palatability purposes (e.g., to obscure unpalatable taste of fish oil), however, and there is no association with the relief provided by the APA. Under the present method, the NPA can be incorporated into, or coated onto a capsule that is then used to contain materials including the APA. One of the advantages of this approach is that different medicines that produce the same or very similar result, or variable dosages of one medication, can use the same type of capsule having a particular sensory cue. The present method is not limited to gelatin-type capsules, and can have an NPA included: 1) with the contents of the APA; 2) as a coating on an APA pill surface; 3) within a second outer capsule containing both the APA in its original form (e.g., pill) in addition to an NPA; 4) within the composition of the second capsule; or 5) as a surface coating on the second outer capsule.

In those applications wherein the NPA is incorporated into, or coated on, a capsule, that same capsule can be used to encapsulate a previously manufactured pill or capsule. In this manner, a sensory cued placebo could be readily used in a prescribed course of treatment as described herein with any APA/medication. This application provides substantial utility because the placebo sensory cue can be added by different stages in the production/administration of the medicine; e.g., added by the original manufacturer of the medication, or by a second-party company to previously manufactured medicine, or by a pharmacist filling a prescription with instructions to add a placebo cue—potentially within a course of treatment wherein the dosage of the APA is reduced over time.

Under the present method, the amount of the APAs within each periodically administered pill is varied over a prescribed course of treatment. In some embodiments, the amount of APA is decreased over a period of time. The present method is not limited, however, to embodiments wherein the amount of the APAs is decreased over time, and the amount of APA may be otherwise varied as the signs or symptoms of the illness for which it is administered vary, and as the physician or health care provider direct.

The sensory cue provided (or caused by) the NPA in each pill enhances the action of the APA. As the amount of APA is gradually decreased within each pill over time, the effect of the NPA proportionately increases and in some cases may become the predominant healing factor. The present method is not limited to a particular manner of decreasing the APA dosage; e.g., the decrease may be linear, or a step function decrease, etc. As the amount of APA is decreased, the sensory cue provided by the NPA contained within each pill remains substantially constant and gives the subject the impression that each pill is in fact the same. In most embodiments of the present method, the amount of the APA within each pill is decreased over time until a lower limit of APA is reached. At that point, each pill will contain the lower limit amount of APA so the subject is always taking some amount of APA during the prescribed course of treatment. The decreased dosage of the APA will result in a significant decrease in APA associated side effects, such as nausea, diarrhea, metabolic disorders, and others.

In the aforementioned manner, patients taking the prescribed medicine can benefit from both the pharmacological action of the APA and the enhanced “placebo effect” of the NPA. The NPA placebo effect will be enhanced because it is associated (both consciously and unconsciously) with a true pharmacologic effect. The physician may prescribe a medication course of treatment wherein the amount of APA tapers down (e.g., “x” percent decrease per day) with no change in the number of pills prescribed each day. Maintaining the prescription at a fixed number of pills likely decreases the potential for medication dosing errors; e.g., applications where the amount of APA is decreased by decreasing the number of pills taken, leading to errors in the number of pills to be taken. In many instances, the patient can continue to take the medication containing the lowest dose of APA to which the disorder being treated is responsive. Maintaining the APA at a very low dose (but still requiring a prescription) also provides the psychological advantage of the subject being under a doctor's supervision and thereby enhancing the salutary effect of the doctor patient interaction and strengthening the placebo response. The supervision helps reinforce the expectation that the course of treatment is significant in providing relief. In other embodiments, the amount of APA within each periodically administered amount of medicine is below the lowest dose with a known pharmacological effect, in which case the dosage of APA may be considered to be a non-active amount of the APA.

The present method for periodically administering a medicine containing one or more active pharmaceutical agents to a patient within a prescribed course of treatment utilizes packaging that facilitates the administration of the medicine. For example, the medicine can be dispensed using a blister pack, or other sequence noting dispenser that makes clear the sequence of pills administered to date. This type of packaging is particularly useful for those applications wherein the pills containing the varying levels of APA and constant levels of NPA are visually identical or difficult to differentiate from each other.

Although the invention has been described and illustrated with respect to exemplary embodiments thereof, the foregoing and various other additions and omissions may be made therein and thereto without departing from the spirit and scope of the present invention.

What is claimed is:

1. A method for administering pharmaceutical agents to a subject, comprising the steps of:
providing a course of treatment for a subject, which course includes periodically administering an amount of medicine to the subject, which amount of medicine includes a dose of at least one active pharmacological agent (APA) and an amount of at least one non-active pharmacological agent (NPA), which NPA provides at least one non-visual sensory cue;
varying the dosage amount of the APA within the periodically administered amount of medicine, while the amount of the NPA contained within each periodically administered amount of medicine is provided at a level that maintains the sensory cue at a substantially constant level.

2. The method of claim 1, wherein the dosage amount of the APA within each periodically administered amount of medicine is gradually decreased over the course of treatment.

3. The method of claim 2, wherein the dosage of the APA within the periodically administered amounts of medicine is a pharmacologically inactive dosage of the APA.

4. The method of claim 2, wherein the dosage of the APA within the periodically administered amounts of medicine is below the generally prescribed dosage of the APA.

5. The method of claim 2, wherein the dosage of the APA within the periodically administered amounts of medicine is at the lowest effective dosage of the APA that continues to be effective in the patient taking the combination of APA and NPA.

6. The method of claim 2, wherein the dosage amount of the APA within each periodically administered amount of medicine is incrementally decreased over the course of treatment until a lower limit dosage amount of the APA is reached, at which point each amount of medicine periodically administered thereafter within the course of treatment contains the lower limit dosage of the APA.

7. The method of claim 6, wherein the dosage of the APA within the periodically administered amounts of medicine is a pharmacologically inactive dosage of the APA.

8. The method of claim 6, wherein the dosage of the APA within the periodically administered amounts of medicine is below the generally prescribed dosage of the APA.

9. The method of claim 6, wherein the dosage of the APA within the periodically administered amounts of medicine is at the lowest effective dosage of the APA that continues to be effective in the patient taking the combination of APA and NPA.

10. The method of claim 1, wherein the dosage of the APA within the periodically administered amounts of medicine is a pharmacologically inactive dosage of the APA.

11. The method of claim 1, wherein the dosage of the APA within the periodically administered amounts of medicine is below the generally prescribed dosage of the APA.

12. The method of claim 1, wherein the dosage of the APA within the periodically administered amounts of medicine is at the lowest effective dosage of the APA that continues to be effective in the patient taking the combination of APA and NPA.

13. The method of claim 1, wherein the periodically administered amounts of medicine are in a form where each administered amount appears to be at least substantially to the other periodically administered amounts, and which form is one of a pill, capsule, tablet, or liquid.

14. The method of claim 13, wherein the periodically administered amounts of medicine are dispensed in a container that indicates a sequential order of the amounts periodically administered.

15. The method of claim 1, wherein the NPA is disposed within a coating applied to one of a pill, capsule, or tablet containing the APA.

16. The method of claim 1, wherein the NPA is incorporated into a capsule that is adapted to encapsulate the APA, which APA is in one of a pill, capsule, or tablet form.

17. The method of claim 1 wherein the NPA is coated on a capsule that is adapted to encapsulate the APA, which APA is in one of a pill, capsule, or tablet form.

18. The method of claim 1, wherein the NPA and APA are combined together and are in one of a pill, capsule, or tablet form.

19. The method of claim 1, wherein the sensory cue provided by the NPA is perceivable by the subject prior to the periodically administered amount of medicine being ingested by the subject.

20. The method of claim 1, wherein the sensory cue is at least one of smell, oropharyngeal somatosensory sensation, and taste.

21. The method of claim 1, wherein the APA administered during the course of treatment creates an effect in the subject, and wherein the sensory cue provided by the NPA is independently associated with the effect caused by the APA.

22. The method of claim 1, wherein the sensory cue provided by the NPA is perceivable by the subject present after the periodically administered amount of medicine has been ingested by the subject.

23. The method of claim 22, wherein the sensory cue provided by the NPA is at least one of a change in color of the subject's urine, a change in smell of the subject's urine, a change in color of the subject's feces, a change in smell of the subject's feces, a change in smell of the subject's breath, an aftertaste in the subject's mouth, a change in smell of the subject's body odor, minor sweating by the subject, or an oropharyngeal somatosensory perception.

24. The method of claim 1, wherein the APA is botanically derived, and the NPA contains one or more non-active constituents from the same or similar species of plants from which the APA was botanically derived.

25. The method of claim 1, wherein the NPA contains one or more non-active constituents from a species of plants known to have pharmacologic actions similar to the desired pharmacologic action of the APA.
